(19) United States
(12) Patent Application Publication
(10) Pub. No.: US 2014/0005537 A1
(43) Pub. Date: Jan. 2, 2014

(54) Photoacoustic Measurement Device

(71) Applicants: Rei Asami, Tokyo (JP); Kennichi Kawabata, Tokyo (JP); Shinta Takano, Tokyo (JP); Takashi Azuma, Tokyo (JP)

(72) Inventors: Rei Asami, Tokyo (JP); Kennichi Kawabata, Tokyo (JP); Shinta Takano, Tokyo (JP); Takashi Azuma, Tokyo (JP)

(73) Assignee: HITACHI, LTD., Tokyo (JP)

(21) Appl. No.: 13/984,460

(22) PCT Filed: Feb. 6, 2012
(86) PCT No.: PCT/JP2012/000756

§ 371 Date: Sep. 18, 2013

(30) Foreign Application Priority Data
Feb. 24, 2011 (JP) 2011-037777

(51) Int. Cl. A61B 5/00; A61B 8/14

(52) U.S. Cl. CPC A61B 5/0095; A61B 8/14
USPC 600/431; 600/407; 600/443

(57) ABSTRACT

A device and an agent for performing imaging in a deep part with high sensitivity and high resolution are to be provided. A photoacoustic measurement device (photoacoustic imaging device) performs light irradiation on an inspection area of a subject to which a photoacoustic agent generating an acoustic signal by causing phase change from a solid phase or a liquid phase to a gas phase by the light irradiation is administered, while the irradiated energy amount is increased, and detects the acoustic signal generated in the inspection area due to the light irradiation to form an imaged image of the inspection area based on the detected acoustic signal.

### TECHNICAL FIELD

The present invention relates to a photoacoustic measurement method and device which visualize information on a subject to which a photoacoustic agent is administered and the like using the photoacoustic agent that generates a characteristic acoustic signal by light irradiation and a photoacoustic measurement device that can distinguish an acoustic signal. Particularly, information on a deep part of a subject using a photoacoustic agent is visualized in detail.

### BACKGROUND ART

When a material is irradiated with optical energy, a phenomenon that a material absorbs the light energy to cause heat generation and thermal expansion, and then, a thermoelastic wave is generated, is referred to as a photoacoustic effect, and the phenomenon has been widely applied to a spectroscopic analysis and in vivo tomography.

For example, when soft tissue in vivo is irradiated with a laser pulse of about several nanoseconds with a wavelength of visible light to infrared light, the light energy is absorbed in a limited volume in the tissue to cause thermal expansion and relaxation and then, a thermoelastic wave is generated. A method of detecting the thus generated acoustic signal, forming an image depending on elapsed time from the light irradiation to the acoustic signal detection, and then, visualizing a structure in vivo is photoacoustic imaging.

In general, in an in vivo diagnostic imaging method, a certain physical property (in a case of X-ray CT, a difference in an X-ray absorbed amount, and in a case of echo, a difference in acoustic impedance) is detected and imaging is performed. Even in a case of photoacoustic imaging, since a difference in the absorption coefficient of light having a specific wavelength is reflected in a difference in an acoustic signal to be generated, in terms of performing imaging on a difference in light absorption, a physical property is detected and imaging is performed. In comparison with optical imaging in which the difference in light absorption is imaged with the reflected light amount, the photoacoustic imaging has an advantage of not being easily influenced by light scattering to perform reception with the acoustic signal. In addition, in comparison with ultrasonic imaging in which transmission and reception are performed using the acoustic signal, the photoacoustic imaging is excellent in that properties on which optical absorbing properties of molecules are reflected can be imaged.

There are various applications and for example, as disclosed in NPL 1, photoacoustic imaging has been used in mammography for detecting breast cancer in which new blood vessels are dense, by observing hemoglobin absorption. More recently, as disclosed in NPL 2, photoacoustic imaging using inexpensive semiconductor laser has been also reported, and it is considered that the photoacoustic imaging will increasingly widely spread more and more.

The photoacoustic imaging has various possibilities as described above. However, while the photoacoustic imaging is influenced by light scattering corresponding to only a half of light scattering of optical imaging, there is still a problem that imaging with high sensitivity is not easily performed in a deep part. As a solution, it is considered that the simplest approach is to increase a light irradiation amount and to increase a light irradiation amount by widening a time width of the pulse.

On the other hand, as disclosed in NPL 3 and PTL 1, an approach in which a deeper part is selectively imaged using a contrast agent is also widely used. This approach is a method in which heat is locally increased in an area where the contrast agent is present by using a metal particle or a pigment having a high absorption coefficient with respect to a specific wavelength, resulting in increasing a thermoelastic wave generated from surrounding tissue.

### CITATION LIST

### Patent Literature

PTL 1: JP-A-2009-137950

### Non Patent Literature

NPL 1: Emirov, S., et al, Proc SPIE, 6086 (2006) 1-12
NPL 2: Allen, T., et al., Proc. SPIE, 5696 (2005) 233-242
NPL 3: Wang, X., et al., Optics Letters, 29 (2004) 730-732
NPL 4: Valluru, K., et al., Ultrasound Clin, 4 (2009) 403-429

### SUMMARY OF INVENTION

### Technical Problem

However, even when the above-described approaches are used, imaging with high sensitivity in a deep part is not easily achieved for the following reasons.

First, in the case of simply increasing the intensity of the light which is irradiated onto a target area to obtain sensitivity, laser intensity to be irradiated is limited by the mechanical limitation of the laser. Further, in terms of safety of a subject, the laser intensity to be irradiated per unit volume is limited.

Second, in the case of considering an increase in a light irradiation amount by widening the time width of the laser pulse to increase sensitivity, as pointed out in NPL 4, photoacoustic imaging is influenced by thermal diffusion of tissue and propagation time of a thermoelastic wave and has a problem of low resolution.

Third, in the case of using the approach to perform imaging on a thermoelastic wave from a living body in which thermal absorption is selectively and locally increased in a deep part using a contrast agent, since an acoustic source depends on tissue as in a normal photoacoustic imaging, and photoacoustic imaging is influenced by thermal diffusion of tissue and propagation time of a thermoelastic wave in the same manner as described above, there is a problem that imaging with high resolution in the deep part is not easily performed.

An object of the invention is to provide a device and an agent that perform imaging with high sensitivity and high resolution in a deep part by solving the above problems.

### Solution to Problem

A representative invention of the inventions is as follows: a photoacoustic measurement device that performs light irradiation on an inspection area of a subject, to which a photoacoustic agent generating an acoustic signal by causing an irreversible phase change from a solid state or a liquid state to a gas state by light irradiation is administered, while an irradiated energy amount is increased, detects the acoustic signal generated in the inspection are a by the light irradiation, distinguishes an acoustic signal from the agent, from an acoustic signal from a reference material or living tissue, in the detected acoustic signal, and forms an photoacoustic image of the inspection area based on the distinguished signal.

### Advantageous Effects of Invention

According to the invention, it is possible to perform photoacoustic imaging in the deep part with high sensitivity and high resolution.

### BRIEF DESCRIPTION OF DRAWINGS

FIG. 1 shows an example of a photoacoustic agent according to an embodiment.

FIG. 2 shows an experimental system of the photoacoustic agent.

FIG. 3 shows a test result of an acoustic response when the photoacoustic agent is irradiated with a laser pulse.

FIG. 4 is a schematic view showing response to the light irradiation of the photoacoustic agent.

FIG. 5 shows an example of a photoacoustic measurement device according to an embodiment.

FIG. 6 shows an example of a configuration of a photoacoustic probe.

FIG. 7 is a schematic view showing a relationship between an acoustic signal from the photoacoustic agent and light irradiation energy per unit volume.

FIG. 8 is a schematic view showing an example of a light irradiation pulse sequence and an acoustic response generated by the light irradiation pulse sequence.

FIG. 9 is a flow chart showing an example of an imaging method of information in vivo using the photoacoustic measurement device.

FIG. 10 shows an example of a photoacoustic measurement device according to another embodiment.

### DESCRIPTION OF EMBODIMENTS

Hereinafter, an embodiment of the invention will be described.

First, a configuration of a device according to the invention will be described. In a photoacoustic measurement device shown in FIG. 5, a subject to which a photoacoustic contrast agent is administered is irradiated with a laser pulse to obtain an image of the subject from the obtained acoustic signal. To the subject shown in FIG. 5, a photoacoustic contrast agent is administered and the contrast agent will be described after the description of the configuration of the device.

As shown in FIG. 5, a probe 8, a console 9 and a display 10 are connected to a photoacoustic measurement device main body 7, and further, the photoacoustic measurement device main body 7 includes a laser pulse switch 11, a receiving beamformer 12, an image processor 13, a transmit/receive sequence controller 14 and a receiving signal processor 15.

The probe 8 is a device which transmits a laser pulse to the subject and receives an acoustic signal from the subject, and has a light irradiation unit 16 which transmits the laser pulse satisfying the condition necessary for gasification of the photoacoustic contrast agent according to the invention and an acoustic signal detector 17 which has a bandwidth and sensitivity for receiving an acoustic signal generated by the irradiation of the subject with light. The light irradiation unit 16 may be any light source as long as the light source has a mechanism in which a light energy amount (for example, a pulse length and pulse intensity) is variable, and preferably, a semiconductor laser can be used. The acoustic signal detector 17 preferably has a mechanism such as that of a focused high-bandwidth hydrophone, and has a structure of mechanically or electrically performing scanning on a focal point. The acoustic signal detector may also have a structure capable of electrically performing focusing and scanning by plural arrayed transducers. In the acoustic signal detector 17, as described later, a signal from the living body can be distinguished from a signal from the contrast agent. Further, the distinction between the signal from the living body and the signal from the contrast agent may be performed by the receiving signal processor.

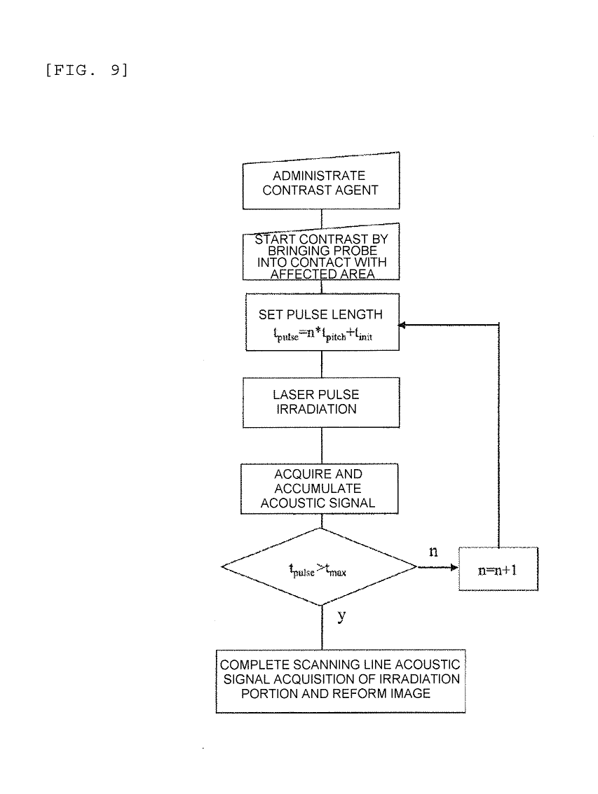

The console 9 is a console necessary for giving various instructions to the photoacoustic measurement device 7. The transmit/receive sequence controller performs control so that the light energy to be irradiated from the light irradiation unit is intermittently increased as shown in FIG. 8(b). The description of FIG. 8 will be made later.

It can be considered that there are 3 examples in the control in which the signal is transmitted from the transmit/receive sequence controller and light is irradiated from the light irradiation unit. One is that an electrical signal which is a control signal to increase the light energy is transmitted to the light irradiation unit 16 from the transmit/receive sequence controller, and the light irradiation unit 16 receives the electrical signal and converts the electrical signal to irradiate light. Here, the control signal is a signal which sends parameters such as a pulse length, a pulse intensity. In the example, the laser pulse switch 11 is not always necessary.

Another example is that the above-described control signal is sent from the transmit/receive sequence controller, the laser pulse switch 11 converts the control signal to a driving signal to drive the light irradiation unit 16, and the light irradiation unit 16 receives the driving signal to irradiate light. Here, the driving signal is a signal which is directly input to a device (here, the light irradiation unit 16) to obtain a desired light output.

The other example is that the light is irradiated from the transmit/receive sequence controller 14, the laser pulse switch 11 turns on or off the timing for light irradiation to perform a process of changing the pulse length, or changing the pulse intensity by changing transmittance using an attenuator. The light irradiation unit 16 irradiates light via the switch 11.

While the 3 examples are mentioned, the invention is not limited to the above-described examples as long as functions of “the controller that transmits an input signal to increase the light energy amount to be irradiated onto an inspection area by repeatedly irradiating the inspection area with the light, and the light irradiation unit that repeatedly irradiates the inspection area with light based on the input signal” are provided from the transmit/receive sequence controller 14.

The inspection area of the subject is irradiated with light from the light irradiation unit 16. Then, the contrast agent present in the inspection area generates an acoustic signal, and the generated acoustic signal (echo signal) is received by the acoustic signal detector 17. The receiving beamformer 12 provides reception directivity to the echo signal. The receiving signal processor 15 distinguishes a tissue component and a contrast agent component as described later. The transmit/receive sequence controller calculates a distance in which the signal is generated based on the elapsed time between the timing of reception of the received echo signal obtained by the receiving beamformer 12 and the timing of the laser pulse irradiation. Finally, the received echo signal is accumulated in the image processor 13, and the image processor synthesizes the images according to the scanning line at the stage of completing electrical or mechanical scanning of one imaging frame, and sends the synthesized image to the display 10 to provide the image as image data.

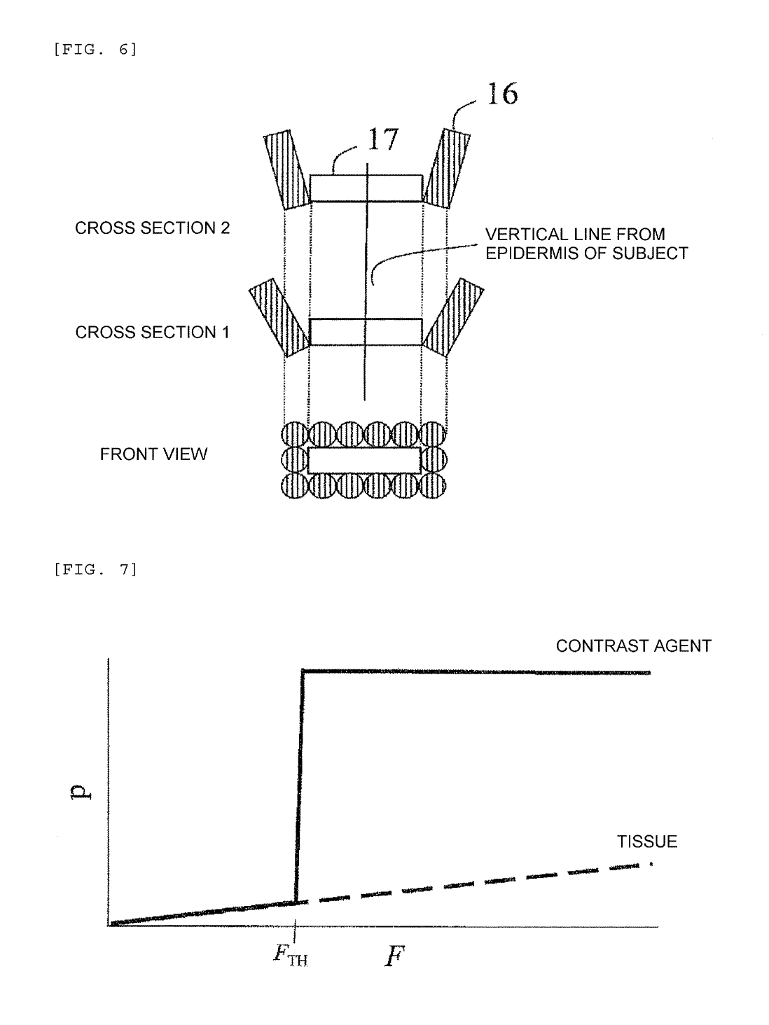

FIG. 6 shows an example of the arrangement configuration of the acoustic signal detector 17 and the light irradiation unit 16 in the probe 8. As shown in the front view of FIG. 6 (a contact surface with the subject), since the light irradiation unit 16 is arranged so as to surround the acoustic signal detector 17, and as shown in the cross-sectional views 1 and 2 (a plane which is vertical to the front view, and the cross-sectional views 1 and 2 show the same plane, but the angle of the light irradiation unit is changed), the angle of the light irradiation unit (as shown in FIG. 6, the angle with respect to a line vertical to the surface of the subject) is variable, directivity can be provided to the light irradiation. Further, the acoustic signal detector 17 and the light irradiation unit 16 do not necessarily form a single probe, and a probe including a light irradiation unit and a probe including an acoustic signal detector may be combined according to circumstances.

Next, the photoacoustic agent used in the invention will be described. The photoacoustic agent used in the invention is a contrast agent which generates an acoustic signal by phase change due to the light irradiation. More preferably, the photoacoustic agent contains at least one poorly water soluble compound which is superheated to a solid state or a liquid state and has a configuration in which an absorbing material having a higher absorption coefficient than that of a reference material or living tissue in at least one wavelength selected from a visible and near-infrared region is attached to the surface of a material which stabilizes the poorly water soluble compound.

The acoustic signal generated from the photoacoustic agent is non-linear to the irradiation light energy and when the photoacoustic measurement device according to the invention is used, the photoacoustic signal can be distinguished from the biological signal. Specifically, the acoustic signal detector or the receiving signal processor in the above-described photoacoustic measurement device distinguishes the contrast agent signal based on an intensity region, a frequency bandwidth limitation, signal discontinuity or the like.

In addition, the photoacoustic measurement device according to the invention includes the transmit/receive sequence controller that controls an irradiation timing and light energy amount (the length of a pulse length or pulse intensity), and the light irradiation can be performed by varying the pulse length and/or pulse intensity. After a phase change from a solid state or a liquid state to a gas state is once caused in the photoacoustic contrast agent, by the light irradiation, the contrast agent is not returned to the state before irradiation and is inactivated to the light irradiation. Therefore, by increasing the light energy amount using the device, the contrast agent present in the superficial part can be sequentially excited from the contrast agent present in the deep part in the subject.

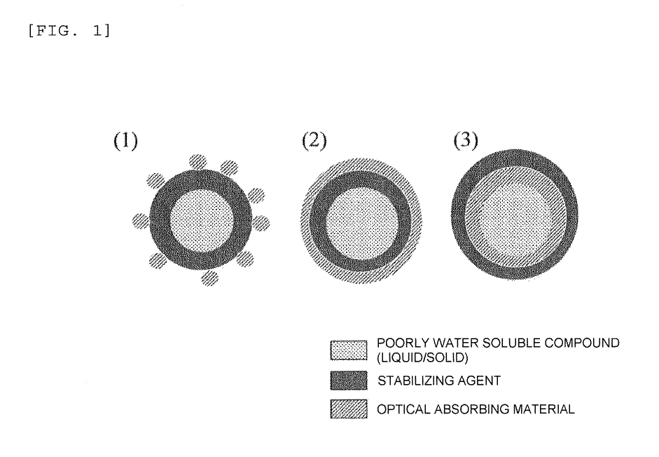

FIG. 1 shows a schematic view of a configuration example of the photoacoustic agent according to the invention. In any of the examples, the photoacoustic agent according to the invention contains three components of (1) at least one poorly water soluble compound which is superheated to solid state or a liquid state, (2) an light absorbing material having a higher absorption coefficient than that of a reference material or living tissue in at least one wavelength selected from a visible and near-infrared region, (3) a stabilizing agent which stabilizes the above-described poorly water soluble compound as main components. Also, the absorbing material is brought into contact with the stabilizing agent.

The poorly water soluble compound which is the first component of the photoacoustic agent according to the invention is a mixture of one or two or more kinds of compatible compounds. When the stabilization is released by the optical absorbing energy from the light irradiation, the compound is instantly gasified. At least one poorly water soluble compound having a boiling point of equal to or less than 37° C. is not particularly limited as long as the compound has biocompatibility. However, preferable examples thereof include linear hydrocarbons, branched hydrocarbons, linear fluorinated hydrocarbons, and branched fluorinated hydrocarbons. Further, when the compound is a mixture of two or more kinds of compounds, at least one poorly water soluble compound having a boiling point of equal to or less than 37° C. and other compounds are preferably materials which have strong intermolecular interactions and cause an azeotropic phenomenon in which the latter is gasified along with the gasification of the former.

The optical absorbing material which is the second component of the photoacoustic agent according to the invention is an absorbing material having a higher absorption coefficient than that of a reference material or living tissue in at least one wavelength selected from a visible and near-infrared region. The absorption wavelength of the optical absorbing material is a wavelength to be emitted during the light irradiation. Specifically, the optical absorbing material is preferably an optical absorbing material which has a large molecular absorption coefficient (ε) and easily transfers excitation energy from an excitation state to other molecules, and more preferably has biocompatibility. More specifically, metal complexes, metal fine particles, organic pigments, synthesized particles and the like are preferred.

As described above, a stabilizing agent having amphipathic properties is necessary for the poorly water soluble compound which is a signal generation source in the photoacoustic agent according to the invention, as particles in a solution and a living body without being dissolved in water. Specifically, as the stabilizing agent, which is the third component of the photoacoustic agent according to the invention and stabilizes the poorly water soluble compound, there may be materials with amphipathic properties such as surfactants, high-molecular compounds (polymers), protein and phospholipid and particles which have biocompatibility and properties that the layer structure is destroyed along with rapid volume expansion of the particles.

In the configuration of FIG. 1 (1), a particulate optical absorbing material is bonded onto the surface of the stabilizing agent, particularly in the case where the optical absorbing material is of fine particles in the form of a polymer, a bonded body or an aggregated body of plural molecules. The structure is particularly effective for a synthesized particle such as a colloidal metal particle (particularly, gold, silver and the like) and a quantum dot. In the configuration of (2), a hydrophilic optical absorber is bonded to a hydrophilic group of the stabilizing agent, and finally encloses the entire particle in layers. The structure is particularly preferable for a hydrophilic optical absorbing material. In the configuration of (3), a lipophilic optical absorber is bonded to a hydrophobic group of the stabilizing agent and is finally embedded in layers inside the stabilizing agent. The structure is particularly preferable for a lipophilic optical absorbing material.

The particle size distribution of these particles is not particularly limited as long as the particle size distribution falls in the range with biocompatibility. Preferably, in angiography such as blood vessel inspection by intravenous injection and lymph vessel inspection by intramuscular injection, a particle size distribution of 1 to 10 μm is effective and in tumor imaging outside a blood vessel, a particle size distribution of 100 to 1000 nm is effective.

The photoacoustic agent according to the invention is useful as a contrast agent for a target affected area by the bonding of molecules (markers) which recognize disease-specific molecules, although not shown in the drawing. For example, new blood vessels around a tumor, vulnerable plaque by arteriosclerosis and the like can be imaged using markers that recognize protein and sugar chains, which are specifically expressed by a disease. Examples of the markers include antibodies and peptide chains. These markers are directly bonded to the stabilizing agent or added to the photoacoustic agent with intermolecular affinity such as polymer or avidin-biotin bonding.

Hereafter, a specific preparation example of the photoacoustic agent will be described. In the example, the contrast agent includes the following composition.

Dipalmotoyl Phosphatydilcoine [1 mM]
Dipalmotoyl Phosphatidic Acid [0.2 mM]
Distearoylphosphatidylethanolamine-5000 PEG [0.2 mM]
Lissamine rhodamine B
1,2-dihexadecanoyl-sn-glycero-3-phosphoethanolamine [1 mM]
Perfluoropentane (PFP) [4% (v/v)]
Perfluorohexane (PFH) [4% (v/v)]
PBS pH 7.4[20 mL]

A test tube containing lipid dissolved in chloroform was put in a water bath at 35° C. and the chloroform was removed under reduced pressure to obtain a thin lipid membrane. Then, 20 mL of PBS was added thereto and a lipid liposome solution was obtained by an ultrasonic fragmentation process. To the liposome solution, PFP and PFH were further added and the mixture was emulsified by a normal-pressure homogenizer. The particle size distribution of the obtained emulsion was measured using a laser diffraction and scattering particle size distribution measurement apparatus (LS13-320, manufactured by Beckman Coulter Inc.), and it was confirmed that the obtained emulsion has a monodispersed distribution with an average particle size of 6 μm. Further, it was confirmed that a pigment is attached using a spectrofluorophotometer.

In addition, in order to obtain a submicron-size emulsion, after the above-described normal-pressure homogenization step, the particles were subjected to a high-pressure emulsification process at a pressure of 150 kpsi using a high-pressure homogenizer and finally obtained a particle size distribution of 150 nm.

In both preparation methods, it was confirmed that the average particle size of the particles can be varied by changing the ratio of the lipid content to the PFP and PFH content.

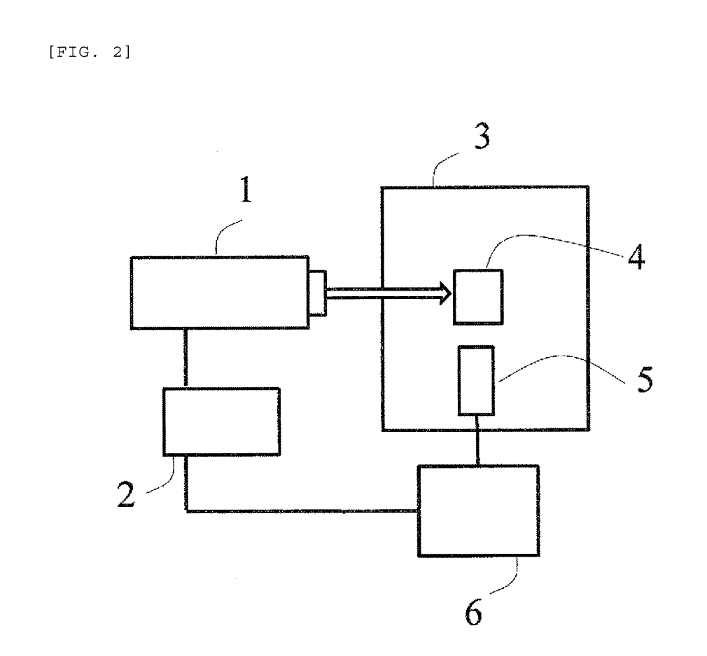

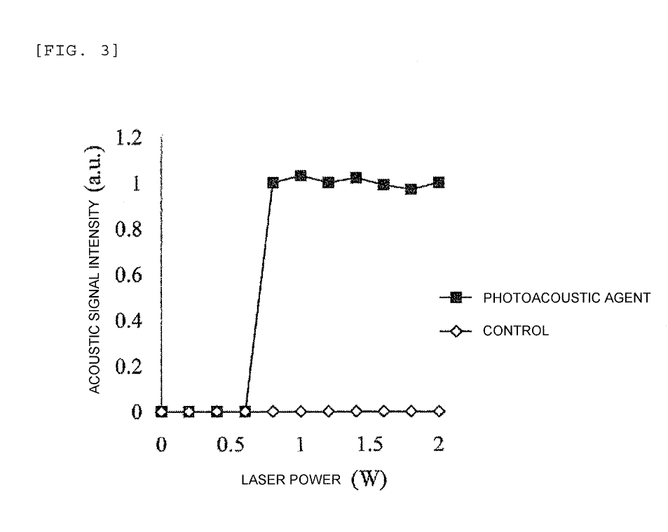

Next, a test example to show the acoustic signal generation during the light irradiation of the photoacoustic agent according to the invention will be described with reference to FIGS. 2 and 3.

FIG. 2 shows an experimental system to perform a test. The experimental system includes a pulse laser 1, a laser driver 2, an optically transparent water bath 3 filled with degassed water at 37° C., a contrast agent sealing phantom holder 4, a hydrophone 5 and an oscilloscope 6. An acrylamide gel phantom containing the prepared photoacoustic agent was left in the water bath, and was irradiated with a pulse laser (Nd:YAG SHG, λ=532 nm, time average power 0 to 2 W). The laser driver and the oscilloscope were synchronized and an acoustic signal was acquired from the focused hydrophone. Further, there was used an agent having exactly the same configuration as that of the photoacoustic agent except that the optical absorbing material is not contained for control. An example of the obtained result is shown in FIG. 3. Moreover, all of the data are obtained from the difference of a sample. After 0.8 W, while in the photoacoustic agent, a strong acoustic signal was observed, in the control which does not contain the optical absorbing material, even at the maximum power, the acoustic signal was not observed at all. Therefore, it is confirmed that the acoustic signal is generated through a mechanism by light absorption action of the optical absorber during the light irradiation of the photoacoustic agent according to the invention.

The above-described test could obtain substantially the same result as in the test review, even when pentane and 2H,3H-perfluoropentane were used instead of using perfluoropentane, and hexane, heptane, octane, pentane, perfluoropentane, perfluorooctane, perfluoroheptanes, perfluorooctanebromide and perfluorodecane are used instead of using perfluorohexane. Furthermore, as the optical absorber, even when the photoacoustic agent to which fine particles such as gold particles and quantum dots, metal complexes such as insulators of porphyrins and phthalocyanines and pigments such as thionine and rose Bengal, instead of the above-described phospholipid with rhodamine, was used, substantially the same result as in the test review could be obtained.

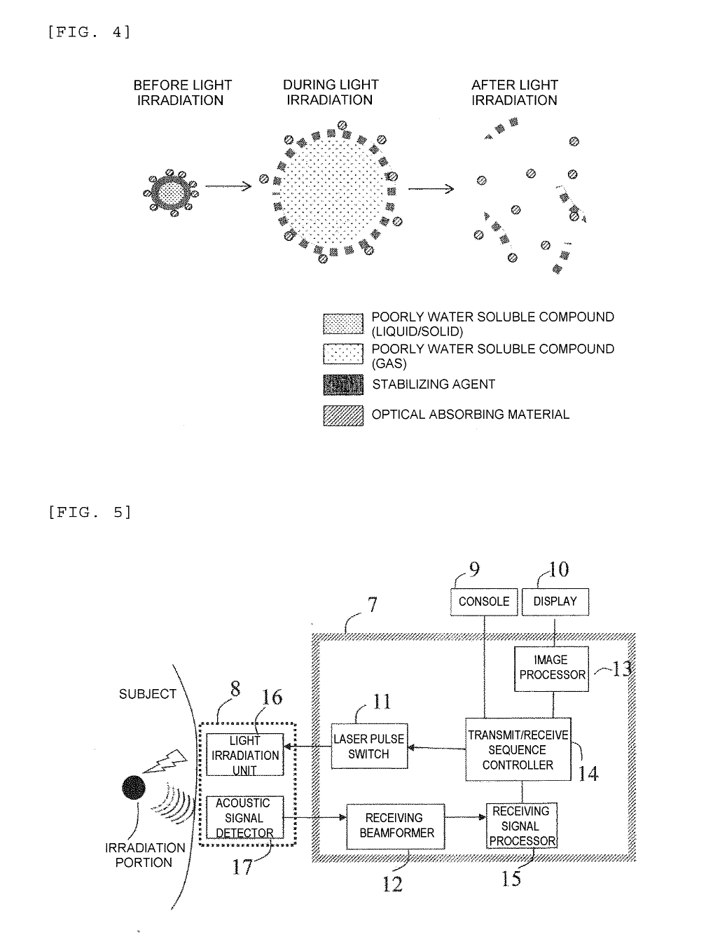

FIG. 4 shows a schematic view showing the response to the light irradiation of the photoacoustic agent according to the invention (non-reversible contrast agent).

First, the optical absorbing material absorbs light by light irradiation and excitation energy is transferred to the stabilizing agent to destroy the stable state of the tensile strength of the stabilizing agent. Then, the liquid or solid state of the poorly water soluble compound is changed to a gas state to generate an acoustic response. At this time, the surface area is increased along with the volume expansion and the stabilizing agent cannot cover the particle. Accordingly, the foamed contrast agent itself cannot be stably present as is clear in the drawing after the light absorption. That is, the acoustic signal is not generated even when the light irradiation is performed again.

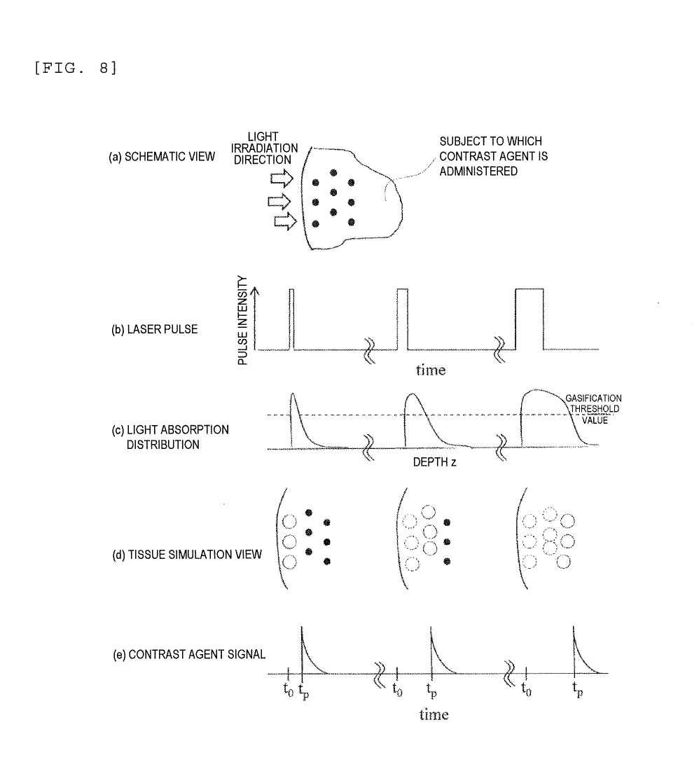

With reference to FIG. 7, the relationship among the acoustic signal from the photoacoustic agent according to the invention, the acoustic signal from the living body and light energy will be described. The photoacoustic signals from tissue and an optical absorbing contrast agent of the related art are derived from thermal expansion caused by an increase in temperature due to the energy absorption of the tissue. Accordingly, the acoustic signal intensity has a linear relationship with respect to light energy per unit volume as represented by Equation 1.

$$\begin{matrix}[ Expression 1 ] & \\ p = \frac{B β}{C ρ} F & Equation 1\end{matrix}$$

(p is an acoustic pressure, F is light energy per unit area, B is volume elasticity, β is a thermal expansion rate, C is specific heat, and ρ is density)

This is as schematically shown by the dotted line of FIG. 7.

Contrarily, when the photoacoustic agent according to the invention exceeds the threshold value of the energy until the photoacoustic agent is changed to the gas state, the phase change instantly occurs. The acoustic signal intensity due to the volume change at this time is represented by Equation 2.

[Expression 2]

p=BX(F≧F_{TH}) Equation 2

(X is a volume change rate during the change from a liquid or solid state to a gas state, and F_{TH} is a threshold value of light energy necessary for phase change)

This is schematically shown by the solid line of FIG. 7, and an approximately-constant high-intensity signal can be obtained when the energy is equal to or more than F_{TH}.

The difference between the intensity of the photoacoustic signal generated from the photoacoustic agent according to the invention and the intensity of the photoacoustic signal generated from living tissue and the like will be described. When perfluoropentane is used as a poorly water soluble compound which is contained in the photoacoustic agent, the apparent boiling point of the superheated perfluoropentane is 42° C. Therefore, in terms of a body temperature of 37° C., when the light energy in which the temperature is increased by 5° C. is irradiated, the perfluoropentane is gasified to generate a signal. In the case of 42° C. in atmospheric pressure, when 1 mL of perfluoropentane is changed from liquid to gas, according to the equation of state of ideal gas, the volume is calculated to be 147 mL. At this time, the signal intensity is p=147B. Contrarily, assuming the living tissue has the same physical properties as water, since the biological acoustic pressure is p=0.001B, the contrast agent signal has about 10⁵ times the intensity of the biological signal. In this manner, the photoacoustic signal intensity is far stronger than the biological signal intensity.

Accordingly, it is possible to distinguish only the signal of the photoacoustic agent according to the invention by setting an arbitrary threshold value of signal intensity and excluding the biological signal area. It is preferable that various signal threshold values be set in advance at the time of device design and an operator can select and finely adjust the threshold values. The distinction method is realized, for example, such that the sensitivity of the acoustic signal detector 17 is lowered. Alternatively, the method is realized by the receiving signal processor 16 such that a signal of equal or lower than a predetermined intensity is omitted.

In the method, the process can be performed using a simple filter and there are merits of a simple signal process and a simple calculation. Moreover, the method is excellent in real time properties.

In addition, as another distinction method, there is a signal distinction method using discontinuity. As is clear from FIG. 7, the photoacoustic agent signal has discontinuous properties that the signal intensity is rapidly increased when the agent is irradiated with a predetermined light energy. Accordingly, plural pulses are continuously irradiated while the energy amount is increased, and only the changes in the discontinuous signal intensity are imaged. Therefore, it is possible to distinguish only the photoacoustic agent signal. That is, a filter which determines discontinuity is mounted on the acoustic signal detector or the transmit/receive sequence controller and extracts the photoacoustic agent signal when discontinuity is determined.

Compared to the above-described intensity difference method, where a threshold value is not easily set in advance in some cases, the discontinuity method has a merit of not having to set a threshold intensity, since discontinuity occurs necessarily.

In particular, when the deep part of the living body is measured, the biological signal intensity is low, and therefore, in the case of using the intensity difference, it is difficult to distinguish noise and the biological signal. However, in the case of determining discontinuity, since discontinuity surely occurs even in the deep part, it is considered that the method is excellent. The distinction method is realized such that the filter which determines discontinuity is mounted on the acoustic signal detector 17 or the receiving signal processor 15.

Further, as the other distinction method, there is a distinction method using a frequency difference. When the time width of the laser pulse is widened, the biological signal is influenced by the thermal diffusion of tissue and propagation rate of the generated acoustic signal, which can be ignored as long as the pulse width is several nanoseconds, and widened to mainly have low-frequency components. Contrarily, even when the time width of the irradiation laser pulse is widened, the frequency properties of the photoacoustic agent signal generated when gasification is caused are not changed. That is, the photoacoustic agent signal can be distinguished by imaging only high-frequency components. In the above-described distinction method using the intensity difference, when there is a material which strongly reflects light in vivo, the distinction is difficult in some cases. However, since there is no such limitation, the distinction method using the frequency difference is excellent and also excellent in real time properties. The distinction method is realized such that a filter which determines the frequency difference is mounted on the acoustic signal detector 17 or the receiving signal processor 15.

The distinction of the contrast agent signal having the above-described 3 properties can be performed by any of the acoustic signal detector 17 and the receiving signal processor 15 in the photoacoustic detecting device according to the invention, and by an arbitrary combination thereof. For example, first, the signal distinction method using signal discontinuity is applied, the photoacoustic signal intensity at the time of the occurrence of a discontinuous change in the signal intensity is automatically set as a threshold value of the acoustic agent signal, and then, the signal distinction method using signal discontinuity can be changed to the signal distinction method using the intensity difference. Such a change can be made by the operator using the transmit/receive sequence controller 14 via the console 9.

An example of a light irradiation method using the photoacoustic agent and the photoacoustic measurement device according to the invention to visualize the deep part with high sensitivity and high resolution will be described with reference to FIG. 8. A subject to which the photoacoustic agent is administered is irradiated with light from the body surface to detect an acoustic signal ((a) in the drawing). The photoacoustic measurement device has the light irradiation unit in which an energy amount (pulse length, pulse intensity and the like) is variable. In the example shown in FIG. 8 (b), particularly, in the light energy amount, the pulse is changed from long to short in length and irradiation is performed. An example in which the pulse length is increased for an increase in the energy amount is described below. However, the embodiment is not merely limited to the increase of the pulse length as long as the light is irradiated to increase the light energy amount as the embodiment.

As shown in (b) of the drawing, in the invention, irradiation can be performed in the order of the pulse length from a short pulse to a long pulse. The initial short pulse is absorbed into the superficial part of the tissue (the left in (c) of the drawing) and only the nearest photoacoustic agent is foamed (the left in (d) of the drawing) and gasified to generate the photoacoustic agent signal (the left in (e) of the drawing). Next, when the light with a long pulse length is irradiated, the energy reaches the deep part while being absorbed nearby. Since the near photoacoustic agent is already inactivated, only the photoacoustic agent present in the deep part is gasified (the center in (d) of the drawing) to generate the acoustic signal (the center in (e) of the drawing). A distance z to a signal generation source in the depth direction is calculated from the elapsed time from the laser pulse irradiation to the acoustic signal detection as follows.

z=c(tₚ−t₀) Equation 3

(c is an acoustic velocity, t₀ is timing of laser pulse irradiation, and tₚ is timing of acoustic signal detection)

As described above, while the pulse length is increased, an irradiation portion is irradiated repeatedly with the pulse, and the information on the calculated distance z is added to the detected acoustic signal. Therefore, it is possible to obtain a photoacoustic image of the deep part with high sensitivity and high resolution.

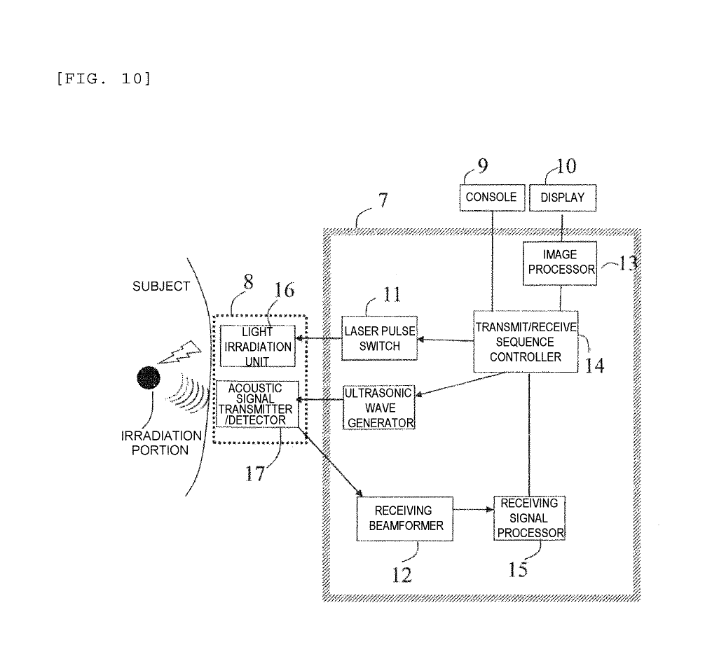

A specific irradiation example of the above sequence will be described in detail with reference to the flow chart in FIG. 9. The light is started to be irradiated onto the subject to which the photoacoustic agent is administered from the short pulse. An initial pulse length tᵢₙᵢₜ, a pulse length amplification pitch t_{pitch} and a maximum pulse length tₘₐₓ may be set at the time of the design of the photoacoustic measurement device, or an operator may set these values to the optimum values through fine adjustment. The initial pulse may have the minimum pulse length which can be mechanically irradiated. In addition, the initial pulse is set to have the length of the pulse irradiated at the time when the pulse with a short length is irradiated and the contrast agent signal is returned, and the pulse length may be increased in each pulse irradiation. The maximum pulse length may be the maximum length of the pulse which can be mechanically irradiated and may be set by an operator.

While the acoustic signal is acquired in each pulse irradiation, a long pulse length is set for each pulse length amplification pitch, and irradiation is sequentially performed. The long pulse is not set in each pulse irradiation, and after some of the same pulses are irradiated, the pulse length may be amplified. Even with the same pulse length as long as the width has a certain degree of depth, it is considered that light reaches the contrast agent. That is, as shown in FIG. 8 (d), even when the leftmost contrast agent is destroyed and then, the light with the same pulse length is irradiated, it is considered that the light reaches the next contrast agent. However, when the optical absorbing material of the contrast agent is destroyed, scattered and is present in vivo, the light does not reach the contrast agent. Therefore, when a certain degree of pulse is irradiated, the pulse length needs to be increased.

Returning to the description of FIG. 9, at the stage of reaching the maximum pulse length, the information on all of the acoustic signals accumulated in the image processor is integrated and the information of the scanning line corresponding to the irradiation portion onto which light is irradiated is calculated to reform an image. Since the acoustic signal of the irradiated portion can be obtained, the number of scanning lines which can be fully processed corresponds to the number of irradiation portions. However, a tomographic image may be obtained such that the information of the scanning lines fewer than the scanning lines which can be fully processed is integrated, and images are reformed. In addition, a tomographic image may be obtained such that plural irradiation portions are set, and images are reformed based on the information obtained from the plural irradiation portions.

The invention is not merely limited to the above-described embodiment and can be embodied in the implementation stage by changing the components thereof within the scope not departing from the gist of the invention. Further, the plural components disclosed in the above-described embodiment can be appropriately combined to form various inventions.

FIG. 10 shows an example of another embodiment of the invention. The embodiment has an ultrasonic wave generator. The controller gives an instruction to the ultrasonic wave generator so as to generate an ultrasonic wave, and a subject is irradiated with the generated ultrasonic wave via an acoustic signal transmitter/detector. The ultrasonic wave generator may simply give an instruction of timing to send an ultrasonic wave, and a wave form to the transmitter/detector, and the transmitter/detector may generate the ultrasonic wave to perform irradiation. In the embodiment of FIG. 10, the light irradiation unit and the acoustic signal transmitter/detector are provided in the same probe. However, the light irradiation unit and the acoustic signal transmitter/detector may be provided in separate probes or may be separate devices. Further, while the transmit/receive sequence controller controls the ultrasonic wave generator in FIG. 10, another controller not shown in the drawing may control the ultrasonic wave generator. In addition, separately from the photoacoustic measurement device shown in FIG. 5, an ultrasonic device may be provided. The ultrasonic device mentioned herein includes at least an ultrasonic wave generator, an ultrasonic probe that transmits and receives an ultrasonic wave to and from a subject, and a processor that processes a received echo signal.

In another embodiment, the above-described light is irradiated and a subject is irradiated with an ultrasonic wave before or after the flow of receiving an acoustic signal. The echo signal from the subject is received by the acoustic signal transmitter/detector and the image processor produces the ultrasonic tomographic image of the subject based on the received signal. Further, the image processor forms an image in which the produced ultrasonic tomographic image and the image produced based on the acoustic signal obtained from the flow in FIG. 9 overlap each other to display the image on the display. From the configuration, an effect of knowing an apparent position of the tumor image produced by the photoacoustic measurement device in the ultrasonic tomographic image which is displayed as a B-mode image is obtained.

### REFERENCE SIGNS LIST

1. Pulse laser
2. Laser driver
3. Optically transparent water bath filled with degassed water at 37° C.
4. Phantom holder
5. Hydrophone
6. Oscilloscope
7. Photoacoustic measurement device main body
8. Probe
9. Console
10. Display
11. Laser pulse switch
12. Receiving beamformer
13. Image reprocessor
14. Transmit/receive sequence controller
15. Receiving signal processor
16. Light irradiation unit
17. Acoustic signal detector

1. A photoacoustic measurement device which irradiates an inspection area of a subject with light to form an photoacoustic image of the inspection area, the device comprising:
a light irradiation unit that irradiates the inspection area with light to which an agent generating an acoustic signal by phase change due to light irradiation is administered;
a controller that controls the light irradiation unit to intermittently increase a light energy amount irradiated from the light irradiation unit;
an acoustic signal detector that detects the acoustic signal generated in the inspection area by the light irradiation; and
an image processor that forms an photoacoustic image of the inspection area based on the detected acoustic signal.

2. The photoacoustic measurement device according to claim 1,
wherein the light energy amount is increased by increasing a laser pulse length.

3. The photoacoustic measurement device according to claim 1,
wherein the light energy amount is increased by increasing pulse intensity.

4. The photoacoustic measurement device according to claim 1,
wherein the controller controls the light irradiation unit to increase the light energy amount in each light irradiation.

5. The photoacoustic measurement device according to claim 1,
wherein the light irradiation unit is a semiconductor laser.

6. The photoacoustic measurement device according to claim 1, further comprising:
a receiving signal processor that extracts a contrast agent signal from the acoustic signal.

7. The photoacoustic measurement device according to claim 6,
wherein the receiving signal processor extracts the contrast agent signal based on the signal intensity of the acoustic signal.

8. The photoacoustic measurement device according to claim 6,
wherein the receiving signal processor determines a degree of the acoustic signal continuity to extract a discontinuous signal as the contrast agent signal.

9. The photoacoustic measurement device according to claim 6,
wherein the receiving signal processor extracts high frequency components from frequency components of the acoustic signal as the contrast agent signal.

10. The photoacoustic measurement device according to claim 1, further comprising:
an ultrasonic wave generator that generates an ultrasonic wave irradiated onto the subject,
wherein the acoustic signal detector further receives an echo signal of the ultrasonic wave, and
the image processor that further produces an ultrasonic image of the subject based on the echo signal.

11. The photoacoustic measurement device according to claim 10,
wherein the image processor forms an image in which the ultrasonic image and the photoacoustic image overlap each other.
